(19) United States
(12) Patent Application Publication
(10) Pub. No.: US 2022/0256886 A1
(43) Pub. Date: Aug. 18, 2022

(54) METHOD FOR PRODUCING COLLAGEN PEPTIDES FROM BONES, AND PRODUCED COLLAGEN PEPTIDES

(71) Applicant: GELITA AG, Eberbach (DE)

(72) Inventor: Ralf PÖRSCHKE, Eschweiler (DE)

(21) Appl. No.: 17/736,430

(22) Filed: May 4, 2022

Related U.S. Application Data
(63) Continuation of application No. PCT/EP2020/077092, filed on Sep. 28, 2020.

(30) Foreign Application Priority Data
Nov. 8, 2019 (DE) 102019130196.3

(51) Int. Cl. A23J 3/34; A23J 1/10; A23J 3/06

(52) U.S. Cl. CPC A23J 3/342; A23J 3/06; A23J 1/10

(57) ABSTRACT

The present invention relates to a method for producing collagen peptides from bones, comprising the following steps: a) providing bones of vertebrates; b) mechanically crushing the bones to a particle size of less than 1 000 μm, preferably less than 500 pm, more preferably less than 300 μm, at a temperature of less than 70° C. during the crushing; c) heating the crushed bones in an aqueous suspension to a temperature of above 100° C., preferably above 120° C., more preferably above 130° C., for a period of from 1 to 30 min, preferably 2 to 10 min, more preferably 4 to 8 min; d) adding one or more proteases to the suspension in order to obtain an aqueous solution of collagen peptides; and e) separating off the aqueous solution of collagen peptides from the crushed bones, wherein the method does not comprise maceration of the bones with an acid or liming of the bones with a base, and wherein the bones provided in step a) have not undergone maceration or liming. The invention further relates to collagen peptides produced by this method.

### CROSS-REFERENCE TO RELATED APPLICATIONS

This patent application is a continuation of International Patent Application No. PCT/EP2020/077092, filed Sep. 28, 2020, which claims the benefit of German Patent Application No. 10 2019 130 196.3, filed Nov. 8, 2019, which are each incorporated by reference in its entirety herein.

### FIELD OF THE INVENTION

Collagen peptides are produced by hydrolysis of the animal structural protein collagen, in particular by enzymatic hydrolysis. Alternative designations are thus collagen hydrolysate or hydrolysed collagen. Where animal bones are the starting material, this is in particular type I collagen.

Collagen peptides are used in various ways in particular in the food industry, on the one hand because of their physiological action in food supplements or so-called functional foods, but also from a food-processing point of view, for example as an emulsifier, stabiliser, binder, etc. A characteristic property of collagen peptides is their solubility, even in cold water, and their poor capacity to form a gel. This enables collagen peptides to be differentiated from gelatine, which is a denatured collagen that is hydrolysed to only a small extent. Collagen peptides have a molecular weight of less than 25 000 Da, indeed usually less than 10 000 Da, while the molecular weights of gelatine are significantly higher.

Collagen peptides are normally produced with gelatine as an intermediate product (see for example R Schrieber and H Gareis: Gelatin Handbook, 2007, Section 2.2.11). In the prior art, the production of gelatine from bones is, for its part, performed in a multistep procedure that comprises as the essential steps the demineralisation of the bones in a strongly acidic medium (maceration) and a subsequent treatment in a strongly alkaline medium (liming), in order then to be able to extract the gelatine at elevated temperature (typically between 50 and 100° C.) in a plurality of steps (see Gelatin Handbook, Section 2.2.5).

During maceration, the roughly ground bones are treated for a period of approximately a week in a counter-current process with dilute hydrochloric acid, in order to elute the mineral components (calcium carbonate and calcium phosphate) from the bone tissue (see Gelatin Handbook, Section 2.2.1.1). The product obtained by this process is called ossein. A cost factor of relevance in maceration, besides the chemicals, is the required cooling because of the exothermic reaction of hydrochloric acid with the calcium minerals. A further disadvantage is the high chloride load in the waste water.

The subsequent liming of the ossein is necessary to enable effective extraction of the gelatine. Typically, the liming process comprises a treatment with a calcium hydroxide suspension (pH value of more than 12) for a period of several months (see Gelatin Handbook, Section 2.2.4.1). Although the treatment time may be shortened by using stronger alkalis (for example to a few days when sodium hydroxide is used), this results in a loss in yield.

The method described above gives type B bone gelatine, which is characterised by an isoelectric point (IEP) of less than 5.6, typically in the range of 4.8 to 5.5. The IEP corresponds to the pH value at which the polypeptide chains of the gelatine (or the collagen peptides produced therefrom) have a neutral overall charge. The relatively low IEP of type B gelatine results from the fact that during liming the amino acids asparagine and glutamine are converted almost entirely to aspartic acid and glutamic acid respectively.

At the same gel strength, type B gelatine has a significantly higher viscosity than type A gelatine, and is therefore preferred for most areas of application. For this reason, the production of type A bone gelatine, in which the ossein is extracted in the acidic medium, without liming, plays only a subordinate role. Type A gelatines have an IEP of greater than 6, in the case of type A bone gelatine typically in the range of between 6 and 8 (in the case of pork rind gelatine in the range of from 8 to 9).

### BRIEF SUMMARY OF THE INVENTION

The present invention relates to a method for producing collagen peptides from bones.

The invention further relates to collagen peptides that are produced by this method.

### BRIEF DESCRIPTION OF THE DRAWING

FIG. 1 shows charge distributions of collagen peptides from the prior art and according to the invention, by means of isoelectric focusing.

### DETAILED DESCRIPTION OF THE INVENTION

The object of the invention is to propose an alternative method for producing collagen peptides, in which the disadvantages of the method described above using type B bone gelatine as an intermediate product can be entirely or partly avoided.

This object is achieved according to the invention with the method of the type mentioned in the introduction in that it comprises the following steps:
a) providing bones of vertebrates;
b) mechanically crushing the bones to a particle size of less than 1 000 μm, preferably less than 500 μm, more preferably less than 300 μm, at a temperature of less than 70° C. during the crushing;
c) heating the crushed bones in an aqueous suspension to a temperature of above 100° C., preferably above 120° C., more preferably above 130° C., for a period of from 1 to 30 min, preferably 2 to 10 min, more preferably 4 to 8 min;
d) adding one or more proteases to the suspension in order to obtain an aqueous solution of collagen peptides; and
e) separating off the aqueous solution of collagen peptides from the crushed bones,

wherein the method does not comprise maceration of the bones with an acid or liming of the bones with a base, and wherein the bones provided in step a) have not undergone maceration or liming.

Within the context of the invention, it was surprisingly found that collagen peptides can be produced by a direct enzymatic treatment of the bone material with proteases without the indirect method by way of producing bone gelatine. Thus, the method according to the invention explicitly dispenses with maceration and/or liming of the bones, as a result of which the overall duration of the method until the collagen peptides are obtained is dramatically reduced: whereas in the extreme case maceration and liming take several months, but at least several days, the method according to the invention can be carried out within a few hours. The energy requirement and the waste water pollution are also significantly less with the method according to the invention than with the prior art.

In the context of the present description, the term “maceration” is understood to mean a treatment with an acid at a pH value of less than 1, and the term “liming” is understood to mean a treatment with a base at a pH value of greater than 12.

As the starting material for the method according to the invention, there may be used in principle bones of any vertebrates, thus including those of birds or fish. Preferably, however, the method is carried out with bones of mammals, in particular bones of bovines.

It is favourable if the bones are cleaned before being crushed, in particular being degreased. Cleaning the starting material favours efficient performance of the enzymatic hydrolysis and makes it possible to produce high-quality collagen peptides.

Preferably, the cleaning of the bones comprises a treatment with one or more enzymes, preferably with proteases and/or lipases. While lipases serve for degreasing, non-collagenous proteins can be broken down and removed using proteases. The proteases hydrolyse the collagen to an only negligible extent before the bones are appropriately crushed.

Before crushing, the bones favourably have a fat content of less than 4 weight %, preferably less than 1 weight %, more preferably less than 0.5 weight %.

As regards the removal of non-collagenous proteins, it is advantageous if, before crushing, the bones have a collagen content of at least 55%, preferably from 70 to 90%, relative to the total protein content. Here, the collagen content is determined from the hydroxyproline content multiplied by a factor of 7.3, and the total protein content is determined from the Kjeldahl nitrogen content multiplied by a factor of 6.25. The said factors take into account the proportion of hydroxyproline and nitrogen in collagen, which differs from the corresponding proportions in the protein as a whole.

The mechanical crushing of the preferably cleaned bones to give a particle size of less than 1 000 μm is an essential feature of the method according to the invention. The small particle size enables direct enzymatic hydrolysis of the collagen in the bone material without the need for the pre-treatments known from the prior art, such as maceration or liming. The mechanical crushing may comprise dry grinding or wet grinding of the bones, wherein wet grinding in an aqueous suspension is preferred. During crushing, the temperature is kept below 70° C. in order to avoid local overheating of the material.

For the purpose of preparing for the enzymatic hydrolysis, the crushed bones are heated to a temperature of above 100° C. in an aqueous suspension, a period of at most 30 min being sufficient for this thermal pre-treatment. During this, the collagen is denatured and made available for the enzymatic hydrolysis. Here, the content of crushed bones by weight in the aqueous suspension is preferably from 0.05 to 0.5 kg/l, preferably from 0.1 to 0.3 kg/l, more preferably from 0.15 to 0.2 kg/l.

Optionally, the thermal pre-treatment of the crushed bones may be further accelerated and/or intensified by an additional input of energy by means of cavitation, for example by ultrasound or a high-pressure homogeniser. Another possibility is to apply AC electrical fields to the suspension.

A further advantage of the method according to the invention consists in the fact that the isoelectric point of the produced collagen peptides can be influenced in a simple manner by adjusting to an appropriate pH value during heating of the crushed bones in the aqueous suspension. Depending on the area of application, collagen peptides with a high or low IEP may be preferred, wherein the differences in the properties are less pronounced here than those of type A and type B gelatine.

In order to obtain collagen peptides with a high IEP of greater than 5.6, before heating the pH value of the aqueous suspension is adjusted to a range of from 5 to 7, preferably from 6 to 7. Typically, a high IEP is produced even if no adjustment of the pH value is made.

In order to obtain collagen peptides with a low IEP of less than 5.6, before heating the pH value of the aqueous suspension is adjusted to a range of from 7 to 9, preferably from 7.9 to 8.6.

After the thermal pre-treatment, the aqueous suspension is cooled to a temperature in the range of from 40 to 60° C. before the addition of the one or more proteases. The optimum activity values of the proteases that are typically used for the enzymatic hydrolysis of collagen lie in this temperature range.

Favourably, the one or more proteases that are added after heating the aqueous suspension are selected from microbial endoproteases, preferably serine proteases, in particular Bacillus subtilis. The use of such enzymes for the hydrolysis of collagen is known from the prior art. A frequently used protease is for example subtilisin.

Typically, the one or more proteases are added in a quantity of from 0.01 to 0.5 weight % relative to the dry mass of the crushed bones, preferably from 0.02 to 0.2 weight %, more preferably from 0.03 to 0.1 weight %.

After the addition of the one or more proteases, the enzymatic reaction is preferably carried out for a period of from 0.5 to 4 hours, more preferably from 1 to 3 hours.

In a preferred embodiment of the invention, once the aqueous solution of collagen peptides has been separated off, the crushed bones undergo steps c) to e) a further time. This twofold heating and treatment of the crushed bones with protease can increase the yield of collagen peptides.

Preferably, separating off the aqueous solution of collagen peptides from the crushed bones comprises a filtration, in particular a membrane filtration. This allows even the smallest particles of crushed bones and other solids to be removed.

After filtration, the aqueous solution of collagen peptides may preferably undergo an ion exchange procedure, in particular salt removal.

According to a preferred embodiment, the method according to the invention further comprises drying the aqueous solution of collagen peptides in order to obtain a collagen peptide powder, in particular by spray drying. The aqueous solution may be concentrated beforehand with the aid of evaporators.

The present invention also relates to collagen peptides that are produced by the method according to the invention.

The collagen peptides according to the invention typically have a weight average molecular weight of less than 25 000 Da, preferably less than 10 000 Da, more preferably less than 5 000 Da. A weight average molecular weight of from 500 to 5 000 Da, in particular from 2 000 to 4 000 Da, is particularly favourable.

In a preferred embodiment of the invention, the collagen peptides have a high isoelectric point of greater than 5.6, in particular greater than 6.0.

According to a further embodiment of the invention, the collagen peptides have a low isoelectric point of less than 5.6, in particular from 5.2 to 5.6.

These and further advantages of the invention become apparent from the examples described below.

### EXAMPLE 1

### Example 1: Production of Collagen Peptides from Bones on a Laboratory Scale

Bovine bones were pre-cleaned by hot water and by protease-supported defleshing and degreasing, and were then ground to a bone powder having a particle size distribution of d50<350 μm and d90<700 μm. The bone powder was then mixed with an equal mass of water and heated to 120 to 130° C. in a microwave oven, with stirring, for approximately 1 min. After cooling (for approximately 20 min) to below 100° C., 0.1 weight % (relative to the bone powder mass) of the protease subtilisin was added and the suspension was stirred at 60° C. Because of the enzymatic reaction with the formation of soluble collagen peptides, the concentration of the aqueous phase rose over time, as indicated in Table 1. The concentration was measured using a refractometer calibrated to the unit ° Brix in order to measure saccharose.

TABLE 1

| | Time/min | | | | | | | |
|---|---|---|---|---|---|---|---|---|
| | 0 | 10 | 20 | 30 | 40 | 50 | 60 | 70 |
| Density/°Brix | 3.4 | 4.9 | 5.2 | 5.3 | 5.5 | 5.5 | 5.5 | 5.5 |

After sedimentation of the bone powder, the supernatant was filtered and the salt removed from it. The collagen peptides were concentrated and dried. The IEP of the collagen peptides was 6.23.

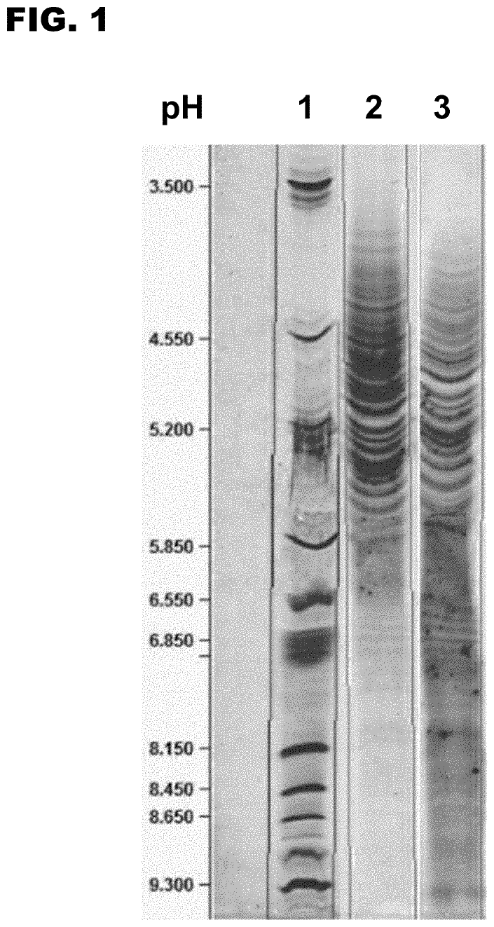

The distribution of molecular charges of these collagen peptides according to the invention can be determined by isometric focusing. FIG. 1 shows a corresponding chromatogram, in which the pH value in the gel is shown on the left and the three tracks are allocated as follows:

Track 1: marker peptides

Track 2: collagen peptides from type B bone gelatine of the prior art (with maceration and liming)

Track 3: collagen peptides according to the invention, in accordance with the example above

The charges of the molecules are generally similar in both samples, with the collagen peptides according to the invention in this case also showing bands of negative—that is to say alkaline—molecules. The pH value on denaturation determines the position of the bands and hence the isoelectric point of the collagen peptides.

### EXAMPLE 2

### Example 2: Production of Collagen Peptides from Bones on a Pilot Scale

An aqueous suspension of 15 weight % of cleaned bones (d90<700 μm) from bovine bone powder was put in a stirred vessel, wherein the bones were cleaned beforehand as in Example 1. The ratio of total protein to collagen was 1.7 (normalised to dry mass less fat content). The pH value of the suspension was adjusted to 6.5.

The suspension was pumped through a heat exchanger by a pump, thus heating it to 130° C. This temperature was maintained for approximately 6 min. Then, the suspension was cooled to approximately 60° C. by a heat exchanger and collected in a stirred vessel. An amount of 0.05 weight % (relative to the dry bone mass) of the protease subtilisin was added. After a reaction time of 2 hours, the enzymatic hydrolysis was ended by heating the suspension to 85° C. for 5 min.

The aqueous phase was separated off using a decanting centrifuge and collected in a vessel. The solid phase was treated in the same way a second time, as described above for the bone powder (production of a suspension, thermal pre-treatment, cooling, enzymatic hydrolysis and separation off of the aqueous phase by decanting).

The aqueous phases (collagen peptide solutions) from the two passes were combined and, for the purpose of further purification, were filtered, underwent salt removal, and were concentrated and dried by suitable methods.

The yield from this method is approximately 16 to 19 weight % of collagen peptides, relative to the bone mass used.

The quality of the collagen peptides may be assessed for example using high transmission values of aqueous solutions having a concentration of 20 weight % at wavelengths of 450 nm and 620 nm. The measured values and the respective quality standard are indicated in Table 2.

TABLE 2

| | Collagen peptides according | Quality |
|---|---|---|
| | to Example 2 | standard |
| Transmission at 450 nm | 74 to 85% | >70% |
| Transmission at 620 nm | 95 to 96% | >95% |

The weight average molecular weight of the collagen peptides according to Example 2 is in the region of 3 000±500 Da. The choice of enzyme, the quantity of enzyme and the reaction time can be used to influence the molecular weight distribution of the collagen peptides according to the invention.

1. A method for producing collagen peptides from bones, comprising the following steps:
a) providing bones of vertebrates;
b) mechanically crushing the bones to a particle size of less than 1 000 μm, at a temperature of less than 70° C. during the crushing;
c) heating the crushed bones in an aqueous suspension to a temperature of above 100° C., for a period of from 1 to 30 min;
d) adding one or more proteases to the suspension in order to obtain an aqueous solution of collagen peptides; and
e) separating off the aqueous solution of collagen peptides from the crushed bones,
wherein the method does not comprise maceration of the bones with an acid or liming of the bones with a base, and wherein the bones provided in step a) have not undergone maceration or liming.

2. The method according to claim 1, wherein the bones come from mammals.

3. The method according to claim 1, wherein the bones are cleaned before being crushed.

4. The method according to claim 3, wherein the cleaning of the bones comprises a treatment with one or more enzymes.

5. The method according to claim 1, wherein, before crushing, the bones have a fat content of less than 4 weight %.

6. The method according to claim 1, wherein, before crushing, the bones have a collagen content of at least 55%, relative to the total protein content, wherein the collagen content is determined from the hydroxyproline content multiplied by a factor of 7.3, and the total protein content is determined from the Kjeldahl nitrogen content multiplied by a factor of 6.25.

7. The method according to claim 1, wherein the mechanical crushing comprises dry grinding or wet grinding of the bones.

8. The method according to claim 1, wherein the heating in an aqueous suspension is carried out with a content of crushed bones by weight of from 0.05 to 0.5 kg/l.

9. The method according to claim 1, wherein, before heating, the pH value of the aqueous suspension is adjusted to a range of from 5 to 7, in order to obtain collagen peptides with an isoelectric point of greater than 5.6, or wherein, before heating, the pH value of the aqueous suspension is adjusted to a range of from 7 to 9, in order to obtain collagen peptides with an isoelectric point of less than 5.6.

10. The method according to claim 1, wherein the aqueous suspension is cooled to a temperature in the range of from 40 to 60° C. before the addition of the one or more proteases.

11. The method according to claim 1, wherein the one or more proteases that are added after heating the aqueous suspension are selected from microbial endoproteases.

12. The method according to claim 1, wherein the one or more proteases are added in a quantity of from 0.01 to 0.5 weight % relative to the dry mass of the crushed bones.

13. The method according to claim 1, wherein, after the addition of the one or more proteases, the enzymatic reaction is carried out for a period of from 0.5 to 4 hours.

14. The method according to claim 1, wherein, once the aqueous solution of collagen peptides has been separated off, the crushed bones undergo steps c) to e) a further time.

15. The method according to claim 1, wherein separating off the aqueous solution of collagen peptides comprises a filtration.

16. The method according to claim 1, further comprising drying the aqueous solution of collagen peptides in order to obtain a collagen peptide powder.

17. Collagen peptides, produced by the method according to claim 1.

18. The collagen peptides according to claim 17, having a weight average molecular weight of less than 25 000 Da.

19. The collagen peptides according to claim 18, having a weight average molecular weight of from 500 to 5 000 Da.

20. The collagen peptides according to claim 17, having an isoelectric point of greater than 5.6.

21. The collagen peptides according to claim 17, having an isoelectric point of less than 5.6.
